(19) United States
(12) Patent Application Publication
(10) Pub. No.: US 2025/0102518 A1
(43) Pub. Date: Mar. 27, 2025

(54) Development of a multiplexing Biosensing platform for the simultaneous detection of Snake and Scorpion venoms

(71) Applicant: Alfaisal University, Riyadh (SA)

(72) Inventors: Abrar AlMusharraf, RIYADH (SA); Amina Rhouati, ALGERIA (DZ); MOHAMMED ZOUROB, RIYADH (SA)

(73) Assignee: Alfaisal University, RIYADH (SA)

(21) Appl. No.: 18/372,555

(22) Filed: Sep. 25, 2023

(51) Int. Cl. G01N 33/68; B82Y 15/00; G01N 33/543

(52) U.S. Cl. CPC G01N 33/6854; G01N 33/54346; G01N 33/5438; B82Y 15/00; G01N 2800/709

(57) ABSTRACT

A dual immunosensor was fabricated on graphene/gold nanoparticle modified screen-printed electrodes and used for simultaneous of the six snake and two scorpion species venoms within wide linear ranges. The electrodes were first modified with two chemical linkers (cysteamine/phenylene diisothiocyanate) in order to facilitate the immobilization of the antibodies through covalent binding. The species specific GPH-GNP/cysteamine/PDITC/SPCE immunosensor was tested with six different snake species venoms and two specific venoms from scorpion species. The detection was observed by monitoring the reduction peak current variation after the venom binding using square wave voltammetry, in presence ferro/ferricyanide redox system.

### FIELD OF INVENTION

A simple and rapid simultaneous detection method for snake and scorpion venom using multiplexing biosensing platform is described.

### BACKGROUND

It has been reported that 1500 different scorpion species have been identified throughout the world; among which 50 are harmful to humans. In Saudi Arabia, 26 different scorpion species have been identified. They belong to four major families including Androctonus crassicauda and Leiurus quinquestriatus species; the most prevalent and highly venomous species. Regarding snake, around 3000 species have been identified worldwide, where 900 were reported as poisonous. In Saudi Arabia, 51 different snake species have been identified, among which nine are poisonous. The venomous snake species belong to different families, including Elapidae (Walterinnesia morgana, Naja arabica, and Walterinnesia aegyptia), Viperidae (Bitis arietans, Cerastes cerastes, Cerastes gasperetii, Echis borkini, Echis coloratus ginther, Echis carinatus, and Pseudocerastes feldi), and Atractaspididae (Atractaspis andersonii and Atractaspis engaddensis). Venom composition varies according to the producing species. In scorpion, venoms are mostly composed of enzymes, proteins, free amino acid, peptides, inorganic compounds, salts, nucleotides, and possibly unidentified substances. Whereas snake venoms include proteins, peptides, lipids, carbohydrate, amino acids, and nucleosides. Treatment of scorpion stings or snakebites envenomation is mainly based on antivenoms administration. They are composed of antibodies developed against venom of one species (Monovalent antivenoms) or venoms of different species (Polyvalent antivenoms). It has been demonstrated that antivenoms administration prevents lethality and the different detrimental envenomation effects.

The diagnosis of snakebites or scorpion stings is usually performed at the hospital through signs and symptoms assessment such as short breath, dizziness and vomiting, tingling, muscle twitching, numbness, swilling, bruising or bleeding around the affected site. If the administration of antivenom is required, healthcare professional should determine which type is appropriate for the patient based on the history and symptoms investigation. In most cases, this is achieved by comparing the clinical characteristics caused by the envenomation against those usually induced by scorpion and snake species. Unfortunately, this is considered as a complicated and inaccurate diagnosis, due to the symptoms interference between snake and scorpion species, which increases the difficulty of identifying the proper treatment to be provided by the health professional. Nevertheless, as far as we know, no commercial diagnostic kits are available for scorpion stings. While for snakebites, Seqirus' Snake Venom Detection kit is only used clinically. It consists in a sandwich immunoassay, specific for tiger, brown, black, death adder and taipan snake species. Recently it was reported a lateral flow immunoassay (LFA) for the detection of Bothrops snake venoms. The developed technique showed a good sensitivity (LOD of 9.5 ng/mL) and a good applicability in urine samples. However, no strategies were developed for the simultaneous detection of scorpion and snake venoms. Therefore, it is still confusing and difficult for the health professionals at emergency rooms to distinguish accurately between snakebites and scorpion stings envenomation where a rapid assessment is necessary to inject the appropriate antivenom.

### SUMMARY

In this study, novel, low cost and easy to use biosensing platform with an assay was developed. In one embodiment, label-free dual immunosensor could be an excellent tool for the management and treatment of snakebites and scorpion stings at emergency room, and in situ analysis without hospitalization. In another embodiment, the multiplexed biosensor was developed on a dual Graphene-Gold nanoparticles modified SPCE composed of two individuals working electrodes. In another embodiment the GPH-AuNPs-SPCE was functionalized by two chemical reagents as linkers (Cysteamine HCL and PDTC). In one embodiment the self-assembled monolayer (SAM) was built by deposition of cysteamine that binds to the surface electrode via its thiol groups. In yet another embodiment, PDITC as a crosslinker, was incubated on the cysteamine modified electrode and in order to enhance the antibodies attachment to the surface by forming a covalent bond between the gold nanoparticles and the thiol group on the linkers.

In one embodiment, both antibodies (Snake and Scorpion antivenoms) were incubated on the surface modified electrodes. In one embodiment, snake and scorpion venoms are mainly composed of proteins, they can interfere with the electron transfer between the redox probe and the electrode surface, thus decreasing SWV signal. In one embodiment, multiplexing immunosensor is designed to distinguish between snake and scorpion envenomation in order to enable faster management and treatment on site and at emergency rooms.

Other features will be apparent from the accompanying drawings and from the detailed description that follows.

### BRIEF DESCRIPTION OF DRAWINGS

Example embodiments are illustrated by way of example only and not limitation, with reference to the figures of the accompanying drawings, in which like references indicate similar elements and in which:

FIG. 1 shows schematic illustration of the fabrication and detection steps of the Snake/Scorpion multiplexed immunosensor.

FIG. 2 shows electrochemical characterization of the modification steps of the immunosensing platform using cyclic voltammetry (CV) at scan rate of 100 mV/s.

FIG. 3A shows optimization of the incubation time for Snake and FIG. 3B shows Scorpion immunosensors.

FIG. 4A, FIG. 4B, FIG. 4C, FIG. 4D, FIG. 4E and FIG. 4F shows Square wave voltammograms recorded for the dual immunosensor against different concentrations of Snake venom solutions (FIG. 4A) Naja arabica, (FIG. 4B) Walterinnesia aegyptia, (FIG. 4C) Bitis arietans, (FIG. 4D) Cerastes cerastes, (FIG. 4E) Echis coloratuts, (FIG. 4F) Echis carinatus.

FIG. 5A and FIG. 513 shows Square wave voltammograms recorded for the dual immunosensor against different concentrations of Scorpion venoms solution of (FIG. 5A) Leiurus quinquestriatus and (FIG. 5B) Androctonus crassicauda.

FIG. 6A and FIG. 6B shows the calibration curves for Scorpion species; (FIG. 6A) Leiurus quinquestriatus and (FIG. 6B) Androctonus crassicauda.

FIG. 7A, FIG. 7B, FIG. 7C, FIG. 7D. FIG. 7E and FIG. 7F The calibration curves plotted for the six snake species (FIG. 7A) Naja arabica, (FIG. 7B) Walterinnesia aegyptia, (FIG. 7C) Bitis arietans, (FIG. 7D) Cerastes cerastes, (FIG. 7E) Echis coloratus, and (FIG. 7F) Echis carinatus.

FIG. 8A and FIG. 8B shows interference studies of the multiplexed immunosensors:

Other features of the present disclosure will be apparent from the accompanying drawings and from the detailed description of embodiments that follows.

### DETAILED DESCRIPTION

In this disclosure, we present a novel, low cost and easy to use portable biosensing platform wherein simultaneous detection of various species venom can be performed. The diagnosis of envenomation accidents is usually performed at the hospital through signs and symptoms assessment such as short breath, dizziness and vomiting, numbness, swilling, bruising, or bleeding around the affected site. However, this traditional method provides inaccurate diagnosis given the interface between snakebites and scorpion stings symptoms. Therefore, the early determination of bites/stings source, would help healthcare professionals in selecting the suitable treatment for patients thus, improving the envenomation management. Herein, we disclose an innovative multiplexing platform based on dual immunosensors for the simultaneous determination of snake and scorpion venoms using label-free electrochemical platform. The dual immunosensor was fabricated on graphene/gold nanoparticle modified screen-printed electrodes. The electrodes were first modified with two chemical linkers (cysteamine/phenylene diisothiocyanate) in order to facilitate the immobilization of the antibodies through covalent binding. The proposed immunosensor was tested with six different snake species venoms and two specific venoms from scorpion species. The detection was undergone by monitoring the reduction peak current variation after the venom binding using square wave voltammetry, in presence ferro/ferricyanide redox system. The dual immunosensor enabled a sensitive and selective simultaneous detection of the six snake and two scorpion species venoms within wide linear ranges. The applicability of the venom immunosensor has been also demonstrated for the detection of snake and scorpion venoms in human serum samples showing high recovery percentages. These achievements show the great potential of our multiplexing approach for the early detection of snake or scorpion envenomation.

Herein, we develop, for the first time, a simple and selective electrochemical immunosensor able to discriminate between snake and scorpion envenomation. Immunosensors are antibodies-based biosensors, which can offer a sensitive, low-cost and fast point-of-care diagnostic for many analytes and diseases, with few steps and without the need for skilled personnel or expensive laboratory infrastructure. Electrochemical detection has gained great attention in last decade due to its robustness, multiplexing capacity, small analyte volumes need, easy miniaturization, and excellent detection limits. Our electrochemical immunosensor consists of a dual graphene/gold nanoparticle modified screen-printed electrode functionalized with two antibodies (polyvalent antivenoms) specific for snake and scorpion venoms, respectively. The polyvalent snake antivenom is specific to six venom species including (Naja arabica, Walterinnesia aegyptia, Bitis arietans, Cerastes cerastes, Echis coloratus, Echis carinatus), while the polyvalent scorpion antivenom is specific to two venom species (Leiurus quinquestriatus & Androctonus crassicauda). The binding between the venom and the immobilized antibody was based on a label-free mode by following the electron transfer decrease using Square wave voltammetry (SWV). The obtained results showed that the immunosensor showed excellent sensitivities toward the different tested species. In addition, the dual immunosensor exhibited a remarkable selectivity owing to the high specificity of employed antibodies toward their targeted venoms. Finally, the immunosensor performance was tested in real serum samples, showing very good agreement with the experiments performed in buffer. We believe that the developed label-free dual immunosensor could be an excellent tool for the management and treatment of snakebites and scorpion stings at emergency room, and in situ analysis without hospitalization.

Reagents and Materials: Cysteamine hydrochloride, 1,4-phenylene diisothiocyanate (PDITC), ethanolamine, N,N-dimethyl formamide (DMF), Potassium ferricyanide (K₃[Fe(CN)₆]), potassium ferrocyanide (K₄[Fe(CN)₆]), were obtained from Sigma-Aldrich (St. Louis, MO, USA). Absolute ethanol and phosphate buffer saline (PBS) were purchased from Sigma (Ontario, Canada). The whole snake and scorpion venoms (Naja arabica, Walterinnesia aegyptia, Bitis arietans, Cerastes cerastes, Echis coloratus, Echis carinatus), (Leiurus quinquestriatus & Androctonus crassicauda) and the antivenoms (antibodies) were obtained from (NAVPC), SA. 1×PBS buffer (sigma-Aldrich), pH 7.4 was used for the venoms and antibodies (snake and scorpion antivenoms) dilutions, as well as washing steps of the immunosensor. Milli-Q water was used for the preparation of all the reagents in this study.

Instrumentation: AUTOLAB potentiostat PGSTAT302N (Multichannel) purchased from Metrohm, Netherlands, was used for all the electrochemical measurements (the cyclic voltammetry (CV) and square wave voltammetry (SWV)). The potentiostat was connected to a computer and carried out by Nova 1.11 software. Graphene-Gold Nanoparticles modified Dual Screen-Printed Carbon electrodes SPCE (C1110GPH-GNP) were obtained from Metrohm DropSens (Spain). Each electrode consists of three parts (two GPH-GNP/Carbon working electrodes. Carbon auxiliary electrode and silver reference electrode). The electrodes were linked to the potentiostat via a specific connector obtained from Metrohm DropSens and are enabled to detect two signals at the same time, allowing (differential) measurement of two samples (snake and scorpion samples solution).

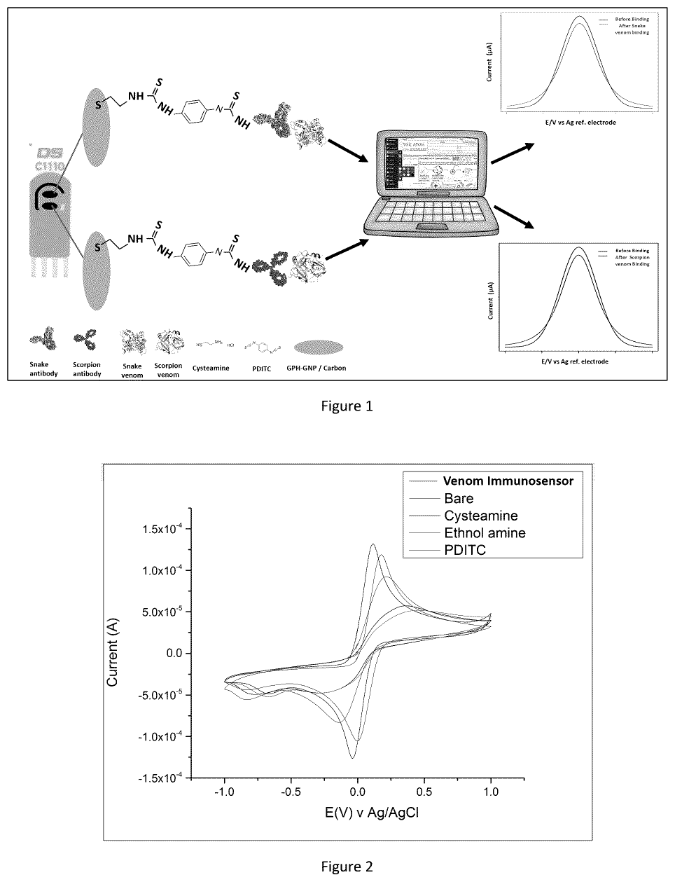

FIG. 1. Schematic illustration of the fabrication and detection steps of the Snake/Scorpion multiplexed immunosensor which are described in detail below.

Fabrication of the dual snake/scorpion immunosensor: Modifying the electrodes and functionalization was achieved by incubating 10 μl of cysteamine hydrochloride (10 mM) on the two working electrodes (GPH-GNP/SPCE), overnight at room temperature. Then, excess residues of cysteamine were removed by rinsing the electrodes with ethanol. Afterwards, 10 mM of PDITC solution prepared in diluted DMF (1:3 DMF) were added to the modified electrodes. After 3 h at room temperature, DMF/H₂O (1:1) and ethanol were used to wash the modified electrodes. Then, conjugating antibodies of a specific species to the modified electrode, such as 5 μl of snake antivenom solution (1:100) and scorpion antivenom solution (1:50) were individually dropped onto the two (GPH-GNP/cysteamine/PDITC/SPCE (W1: Snake Ab and W2: Scorpion Ab) and incubated overnight at 2-8° C. to form a species specific GPH-GNP/cysteamine/PDITC/SPCE immunosensor. Next, the non-specifically bound antibodies were eliminated by washing the electrodes with PBS (pH 7.4). To block the non-specific sites on the surface, a solution of ethanolamine 0.1 M was incubated on the electrodes for 30 min. Finally, the electrodes were rinsed with PBS buffer (pH 7.4) and used for snake and scorpion venoms detection experiments. Cyclic voltammetry was employed for the characterization of the immunosensor fabrication steps.

Detection experiments on the immunosensor: 3 μl of increasing concentrations, ranging between 50 μg/ml and 1400 μg/ml of Snake and Scorpion venoms were incubated with the Snake/Scorpion immunosensors for 30 min at room temperature. Then, the dual immunosensor was rinsed with PBS buffer (pH 7.4), and the square wave voltammograms were recorded in the redox system (5 mM ferri/ferrocyanide prepared in PBS buffer pH 7.4).

Selectivity studies: To study the selectivity of the proposed strategy, each working electrode of the dual Snake/Scorpion immunosensor was incubated 30 min at room temperature with 0.2 μg/ml of the non-specific target. The snake immunosensor was incubated with Leiurus quinquestriatus venom while scorpion immunosensor was incubated with Cerastes ceraste venom. Then the dual Snake/Scorpion immunosensors were washed with PBS buffer and square wave voltammograms were recorded in the redox solution (5 mM ferri/ferrocyanide prepared in PBS buffer pH 7.4). Lastly, the responses were compared to that of the specific target of both snake and scorpion venoms.

Application of Snake/Scorpion immunosensor in real serum sample: For the purpose of evaluating whether Snake/Scorpion immunosensor-based detection can be applied on real biological samples, we used the immunosensor on a non-infected serum samples spiked with snake and scorpion venom solutions. Serum samples were used after getting the approval (IRB: H-02-K-076-00520-298) from the Saudi Ministry of Health. First, serum samples were diluted ten times in PBS buffer, pH 7.4. Then, they were spiked with different concentrations of Bitis arietans (Snake venom species) and Androctonus crassicauda (Scorpion venom species). Afterwards, the spiked samples were incubated with the dual immunosensor for 30 min at room temperature. Finally, SWV measurements were performed after washing with PBS buffer.

The principle of the Snake/Scorpion multiplexed immunosensor: As shown in the schematic diagram at FIG. 1, the multiplexed biosensor was developed on a dual Graphene-Gold nanoparticles modified SPCE composed of two individuals working electrodes. The modified graphene-gold electrode's surface was selected for a better immobilization of the antibodies. The combination of graphene and gold nanomaterials has been often used in electrode surface modifications, because of their numerous advantages. Graphene is highly conductive and provides a large surface area, while Gold is chemically stable and considered as a good conductor (He et al., 2019). The GPH-AuNPs-SPCE was functionalized by two chemical reagents as linkers (Cysteamine HCL and PDITC). At the beginning, the self-assembled monolayer (SAM) was built by deposition of cysteamine that binds to the surface electrode via its thiol groups. Then, the PDITC as a crosslinker, was incubated on the cysteamine modified electrode and in order to enhance the antibodies attachment to the surface by forming a covalent bond between the gold nanoparticles and the thiol group on the linkers (Gautier, López, & Breton, 2021; Shimaa Eissa, 2018). These steps provide a uniform monolayer of well oriented and organized antibodies on the surface, which can improve the sensitivity and selectivity of the immunosensor. It can also provide a stable and reproducible surface for the immobilization of antibodies (Wink et al., 1997). After SAMs steps, both antibodies (Snake and Scorpion antivenoms) were incubated on the surface modified electrodes. The principle of detection was based on the electrochemical signal change upon the specific binding of the targeted venom to its specific antibody (snake and scorpion antivenoms). As snake and scorpion venoms are mainly composed of proteins, they can interfere with the electron transfer between the redox probe and the electrode surface, thus decreasing SWV signal (Das, Saviola, & Mukherjee, 2021; Jenkins et al., 2021; Tasoulis & isbister, 2023: Tasoulis, Pukala, & Isbister, 2021).

Electrochemical characterization analysis: The characterization of the fabrication steps on the surface electrodes including the immobilization of antibodies (Snake and Scorpion antivenoms) were confirmed by using CV measurements, a powerful electrochemical technique often used to study reductions and oxidations phenomenon's (Oberhaus, Frense, & Beckmann, 2020; Wink et al., 1997). CV measurements were performed before and after surface modifications using redox solution (5 mM ferri/ferrocyanide solution prepared in PBS buffer pH 7.4) within a potential range of (−1 to 1 V) and a scan rate of 100 mV/s.

FIG. 2 shows electrochemical characterization of the modification steps of the immunosensing platform using cyclic voltammetry (CV) at scan rate of 100 mV/s. As displayed in FIG. 2, the bare graphene/gold electrode showed quasi-reversible peaks with a peak-to-peak separation (ΔE) around 0.17 mV indicating the high conductivity of the modified graphene/gold surface. Then, a slight increase in the current and decreased at the ΔE around 0.14 mV has been observed after the incubation with cysteamine, indicating the successful self-assembly of the cysteamine on the gold surface via its terminal amine groups that promote electron transfer (Oberhaus, Frense, & Beckmann, 2020). Then, following the reaction cysteamine-PDITC, the current showed a decrease. This phenomenon could be attributed to the negatively charged isothiocyanate groups that hampered transfer of electrons to the surface (Elshafey et al., 2013). Finally, a further decrease in the peak current and increase of the ΔE at around 0.6-0.67 mV, were noticed after the antibodies immobilization and ethanolamine blocking, respectively. This phenomenon could be attributed to the bulky size of antibodies thus inhibiting the transfer of electrons to the electrode surface. These findings are in a good accordance with previous studies reported by our group ((Shimaa Eissa, 2018).

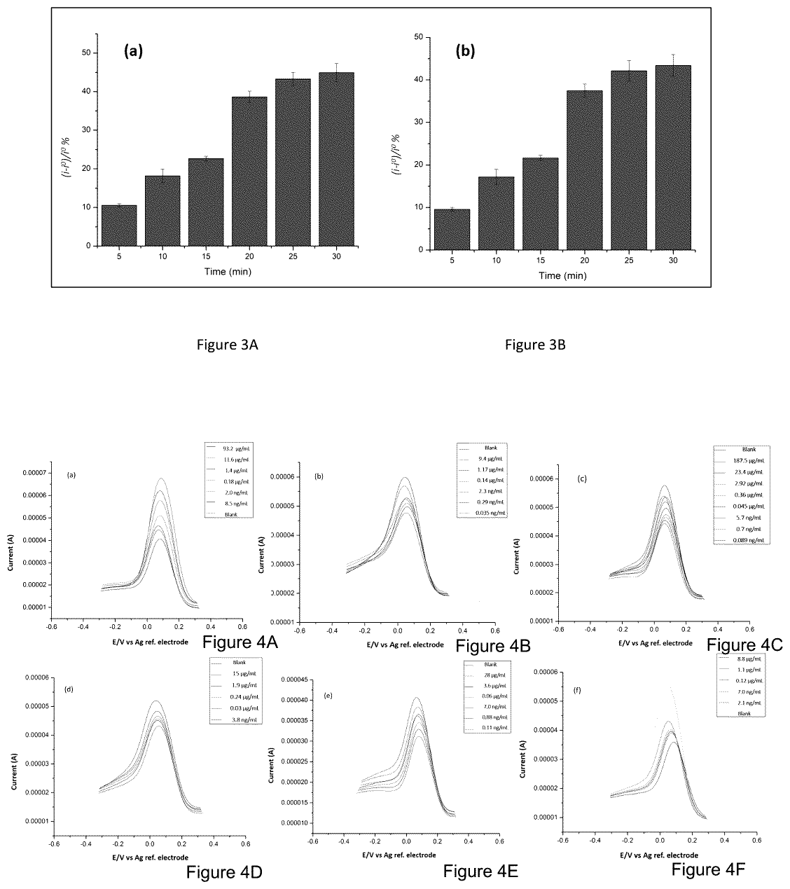

Optimization of the incubation time of the multiplexing immunosensor with the targeted venoms: Binding time between the antigen and its specific antibody is one of the most significant experimental conditions that might influence the performance of immunosensors. For that, different periods of time were tested (from 5 to 30 minutes) to obtain the best response signals on the venom immunosensors. The two functionalized electrodes in the dual platform were incubated with 0.2 μg/mL of the targeted venom, and the obtained responses were calculated based on the decrease of reduction peak currents measured by SWV before and after the formation of the complex antibody-venom. The immunosensor response was determined as the percentage ((i−i⁰)/i⁰%), where i⁰ is the current before, and i is the current after incubation with venom solutions, respectively. The responses of the multiplexed immunosensors at different time points are showed in FIGS. 3a and b, corresponding to Snake and Scorpion venoms, respectively. A gradual increase can be obviously seen for snake and scorpion immunosensors with incubation time. However, the increase was not significant between 20 and 30 minutes for both sensors. Consequently, we choose the optimized incubation time of 30 min to perform the detection experiments.

FIG. 3A and FIG. 3B shows optimization of the incubation time for Snake (a) and Scorpion (b) immunosensors (The sensor response ((i−i⁰)/i⁰%) corresponding to the tested periods of time). The error bars represent the standard deviations of triplicate measurements.

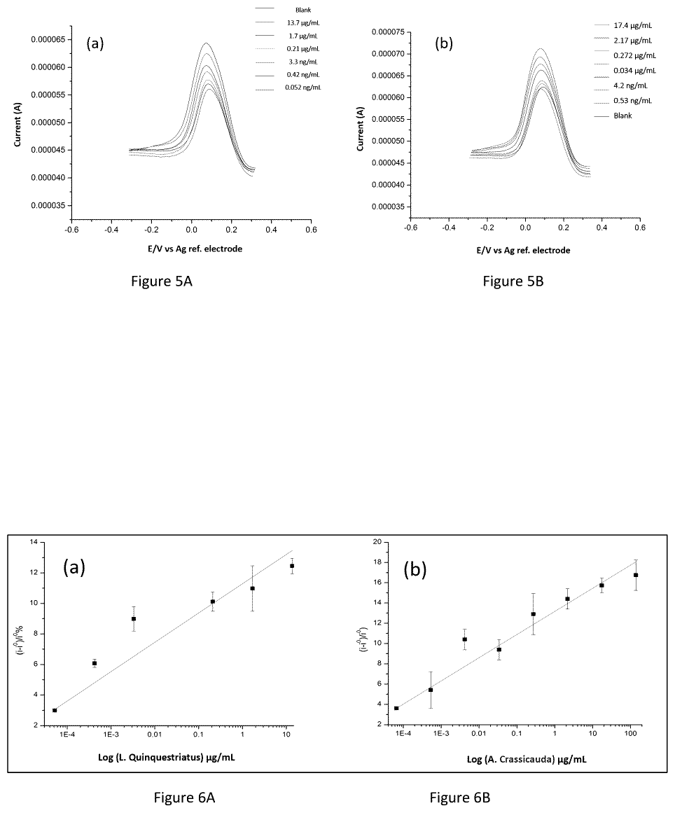

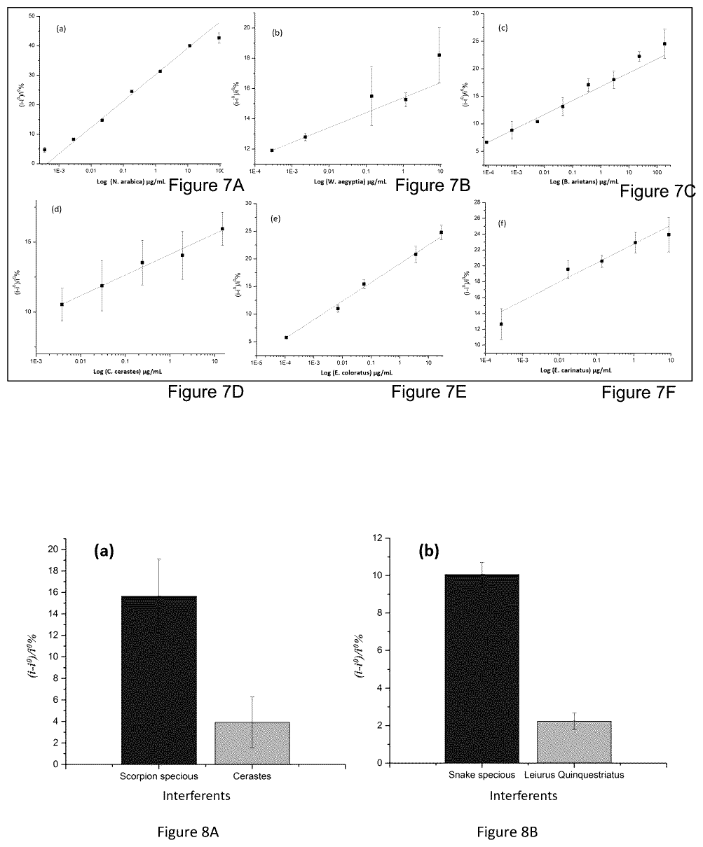

Dose-response of the dual immunosensor for Snake and Scorpion venoms detection: Evaluation of the analytical performance of the venom multiplexed immunosensor was accomplished by testing the dual immunosensor response toward increasing concentrations of snake and scorpion venoms solutions. For that, square wave voltammograms were recorded between 0.3 to −0.3 V, frequency 25 Hz, interval time 0.04 s, step potential −5 mV, scan rate 125 mV/s and amplitude 20 mV, after incubating the immunosensors with the venoms within the range of 50 μg/mL to 1400 μg/mL. FIG. 4A, FIG. 4B, FIG. 4C FIG. 4D FIG. 4E and FIG. 4D displays the SWV results of the dual immunosensor against different concentrations of snake venoms solution (FIG. 4A) Naja arabica, (FIG. 4B) Walterinnesia aegyptia, (FIG. 4C) Bitis arietans, (FIG. 4D) Cerastes cerastes, (FIG. 4E) Echis coloratus, (FIG. 4F) Echis carinatus. SWV results corresponding to scorpion venoms (FIG. 5A) Leiurus quinquestriatus and (FIG. 5B) Androctonus crassicauda. As it can be seen in the FIG. 5A and FIG. 5B, the reduction peak currents decreased by increasing the venoms concentrations for all snake and scorpion species. This is likely due to interaction between the antibodies on the dual immunosensor and their targets snake/scorpion venom proteins (venoms structure complex). As it was expected, the formation of the complex venom-antivenom hampers the electron transfer to the surface. The dual immunosensor responses were determined based on the peak current change after the venoms binding. Then, the calibration curves were plotted using the calculated percentages ((i−i⁰)/i⁰%) as function to the logarithm of the increasing concentrations of venoms. FIGS. 7A, 7B, 7C, 7D, 7E and 7F represents the calibration curves plotted for the six snake species (FIG. 7A) Naja arabica, (FIG. 7B) Walterinnesia aegyptia, (FIG. 7C) Bitis arietans, (FIG. 7D) Cerastes cerastes, (FIG. 7E) Echis coloratus, and (FIG. 7F) Echis carinatus, respectively. Table 1 shows the obtained linear ranges as well as the linear regression and R² for the six tested species. As it can be seen, wide linear ranges were obtained for scorpion and snake species except Walterinnesia aegyptia and Cerastes cerastes, where the ranges were slightly narrower: 35.9 pg/mL-9.4 μg/mL and 3800 pg/mL-15 μg/mL, respectively. This explains the shape of the corresponding calibration curves in FIGS. 7B and 7D. FIG. 6A and FIG. 6B show the obtained calibration curves for the two scorpion species (FIG. 6A) Leiurus quinquestriatus and (FIG. 6B) Androctonus crassicauda. Excellent relationship was obtained for the two species within the range of (52 pg/mL-13.7 μg/mL) for Leiurus quinquestriatus and (66 pg/mL-139 μg/mL) for Androctonus crassicauda. The linear regression was as: ((i−i⁰)/i⁰%)=11.28+1.91 Log Leiurus quinquestriatus (μg/mL) for species Leiurus quinquestriatus with R² equal to 0.92 and ((i−i⁰)/i⁰%)=13.16+2.28 Log Androctonus Crassicauda (μg/mL) with R² equal to 0.98 for species Androctonus crassicauda.

The sensitivity was calculated as 3 σ/b, with the limits of detection LODs: 0.079, 0.021, 0.016, 0.017, 0.012, 0.010, 0.023 and 0.019 μg/mL for species Naja arabica, Walterinnesia aegyptia, Bitis arietans, Cerastes cerastes, Echis coloratus, Echis carinatus, Leiurus quinquestriatus and Androctonus crassicauda, respectively. In order to demonstrate the reproducibility of the proposed multiplexing venom immunosensor, the detection trials were performed three times.

FIG. 4A, 4B, 4C, 4D, 4E, 4F shows Square wave voltammograms recorded for the dual immunosensor against different concentrations of Snake venom solutions (FIG. 4A) Naja arabica, (FIG. 4B) Walterinnesia aegyptia, (FIG. 4C) Bitis arietans, (FIG. 4D) Cerastes cerastes, (FIG. 4E) Echis coloratus, (FIG. 4F) Echis carinatus. SWV performed from 0.3 to −0.3 V, frequency 25 Hz, interval time 0.04 s, step potential −5 mV, scan rate 125 mV/s and amplitude 20 mV.

FIG. 5A and FIG. 5B shows Square wave voltammograms recorded for the dual immunosensor against different concentrations of Scorpion venoms solution of (FIG. 5A) Leiurus quinquestriatus and (FIG. 5b) Androctonus crassicauda. SWV performed from 0.3 to −0.3 V, frequency 25 Hz, interval time 0.04 s, step potential −5 mV, scan rate 125 mV/s and amplitude 20 mV.

FIG. 6A and FIG. 6B shows the calibration curves for Scorpion species; (FIG. 7B) Leiurus quinquestriatus and (FIG. 6A) Androctonus crassicauda. Plots of the sensor response ((i−i⁰)/i⁰%) versus the logarithm of venoms concentrations (μg/mL). The error bars represent the standard deviation of three measurements.

FIG. 7A, 7B, 7C, 7D, 7E, 7F The calibration curves plotted for the six snake species (FIG. 7A) Naja arabica, (FIG. 7B)Walterinnesia aegyptia, (FIG. 7C) Bitis arietans, (FIG. 7D) Cerastes cerastes, (FIG. 7E) Echis coloratus, and (FIG. 7F) Echis carinatus. Plots of the sensor response ((i−i⁰)/i⁰%) versus the logarithm of venoms concentrations (μg/mL). The error bars represent the standard deviation of three measurements.

TABLE 1
Snake venoms detection ranges, the linear regression and R²

| Sanke species | Linear range | Linear regression | R² |
|---|---|---|---|
| Naja arabica | 360 pg/mL- | 30.27 + 8.96 Log Naja | 0.99 |
| | 93.2 μg/mL | Haje Arabicus concentration | |
| | | μg/mL) | |
| Walterinnesia | 35.9 pg/mL- | 11.28 + 1.91 Log Walterinnesa | 0.97 |
| aegyptia | 9.4 μg/mL | Aegyptia concentration | |
| | | (μg/mL) | |
| Bitis arietans | 89 pg/mL- | 14.74 + 2.51 Log Bitis | 0.97 |
| | 187.5 μg/mL | Arietans concentration (μg/mL) | |
| Cerastes cerastes | 3800 pg/mL- | 14.13 + 1.47 Log Cerastes | 0.98 |
| | 15 μg/mL | Ceraste concentration | |
| | | (μg/mL) | |
| Echis coloratus | 110 pg/mL- | 19.14 + 3.38 Log Echis | 0.99 |
| | 28 μg/mL | Coloratus concentration | |
| | | (μg/mL) | |
| Echis carinatus | 0.27 pg/mL- | 22.76 + 2.38 Log Echis | 0.90 |
| | 8.8 μg/mL | Carinatus concentration (μg/mL) | |

FIG. 8A and FIG. 8B shows interference studies of the multiplexed immunosensors: ×(FIG. 8A) Analytical responses of the Scorpion immunosensor to 0.2 μg/mL of Cerastes ceraste venom and scorpion specific venom species (FIG. 8B) Analytical responses of the Snake immunosensor to 0.2 μg/mL of Leiurus quinquestriatus venom and snake specific venom species.

The selectivity investigation of the multiplexed biosensor: As it was mentioned above, the main goal of our multiplexing immunosensor is to distinguish between snake and scorpion envenomation in order to enable faster management and treatment on site and at emergency rooms. Therefore, it is of great importance to demonstrate that our each immunosensor in our dual detection array doesn't present a cross reactivity for the second one. To demonstrate that, each immunosensor was incubated with its specific target as well as the non-specific targeted venom, separately. Then, square wave voltammograms were recorded following the same procedure described above. FIGS. 8A and 8B show the obtained electrochemical responses for scorpion and snake immunosensors, respectively. These responses were calculated as the percentage: ((i−i⁰)/i⁰%)) where i⁰ and i represent the current peak before and after the venom binding, respectively. It can be clearly seen in FIG. 8A that scorpion immunosensor exhibited an electrochemical response toward Cerastes ceraste species 4 folds lower than its response to scorpion species venom at the same concentration of 0.2 μg/mL. Similarly, in FIG. 8b the snake immunosensor response against its target was also significantly 5 folds higher than the response recorded against Leiurus quinquestriatus venom. Based on these findings, we can confirm that the multiplexed immunosensors are highly selective and specific to their targets and can be successfully used to distinguish between the two venoms derived from snake and scorpion bites.

Application of the venoms immunosensor on real samples: In the last step of our study, the applicability of the dual immunosensor to detect and discriminate snake and scorpion infections in real samples was evaluated. For that, blood samples were extracted from non-infected patients. Then, serum was obtained from non-infected patient through centrifugation of the blood samples at 1000 rpm for 3 min, then the serum was diluted 10 times with PBS buffer (PH 7.4). Afterwards, the samples were spiked with different concentrations of snake (0.089, 5.7 and 45 ng/mL) and scorpion (0.066, 0.53 and 4.2 ng/mL) venoms. The dual immunosensor was subsequently incubated with the spiked samples and the electrochemical responses were recorded by SWV. The corresponding percentages ((i−i⁰)/i⁰%)) were calculated and compared to that obtained in buffer. As shown in table 2, the measured concentrations were very close to the added ones with excellent recovery percentages ranging from 95.7% to 110%. These results confirm that the proposed dual venom immunosensor could be successfully applied in infected patients without matrix effects.

TABLE 2
the results of the detection of Snake and Scorpion venoms
on serum samples.

| | Added | Found | Recovery | |
|---|---|---|---|---|
| | concentration | concentration | percentage | RSD |
| Venom | (ng/mL) | (ng/mL) | (%) | (%) |
| Snake | 0.089 | 0.086 | 96.77 | 7.09 |
| | 5.7 | 6.27 | 110 | 2.65 |
| Scorpion | 45 | 46 | 102 | 3.61 |
| | 0.066 | 0.068 | 103 | 1 |
| | 0.53 | 0.519 | 98 | 3.79 |
| | 4.2 | 4.02 | 95.7 | 1 |

In conclusion, an innovative simple and rapid label-free multiplexed immunosensor for the simultaneous detection of Snake and Scorpion venoms was developed. The dual immunosensor was fabricated on graphene/gold modified screen-printed electrodes. Specific antivenoms to six snake and two scorpion venom species were used as bioreceptors for a high affinity detection. The immunosensor responses were determined by measuring the decreasing electrochemical signal by square wave voltammetry after the venoms binding. The venom immunosensors have shown good sensitivities and wide linear ranges. In addition, selectivity studies have shown that the two immunosensors have no cross reactivity for the tested non-specific venoms. Furthermore, we demonstrated the applicability of the multiplexing immunosensor in human serum samples where we obtained a good agreement with experiments performed in buffer solution. The obtained results confirm that our platform allows the successful discrimination of snake/scorpion venoms. Therefore, we anticipate that the multiplexed immunosensor would aid health professionals in the early detection of envenomation at the emergency room.

What is claimed is:

1. A method of detecting a venom in different species simultaneously, comprising:
modifying a Graphene-Gold Nanoparticle (GPH-GNP)-Screen-Printed Carbon (SPCE) electrode with at least two chemical linkers in order to facilitate the immobilization of the antibodies through covalent binding;
washing the electrode with a N,N-dimethyl formamide and water mix and then ethanol to form a modified electrode;
conjugating an antibody of a specific species was added to the modified electrode to form a species specific GPH-GNP/cysteamine/PDITC/SPCE immunosensor;
detecting a species specific venom by adding the species specific venom to the species specific GPH-GNP/cysteamine/PDITC/SPCE immunosensor and incubating in room temperature;
monitoring the reduction peak current variation after the venom binding using square wave voltammetry.

2. The method of claim 1, wherein the linkers are Cysteamine HCL and 1,4-phenylene diisothiocyanate.

3. The method of claim 1, wherein, the species in a scorpion are Leiurus quinquestriatus and Androctonus crassicauda.

4. The method of claim 1, wherein the species in a snake species are Naja arabica, Walterinnesia aegyptia, Bitis arietans, Cerastes cerastes, Echis coloratus, Echis carinatus.

5. The method of claim 1, wherein, the modified electrode is Graphene-Gold Nanoparticle/cysteamine/PDITC/SPCE electrode.

6. A method of detecting a venom in a snake species simultaneously, comprising:
modifying a Graphene-Gold Nanoparticle (GPH-GNP)-Screen-Printed Carbon (SPCE) electrode with at least two chemical linkers in order to facilitate the immobilization of the antibodies through covalent binding;
washing the electrode with a N,N-dimethyl formamide and water mix and then ethanol to form a modified electrode;
conjugating an antibody of the snake species was added to the modified electrode to form a species specific GPH-GNP/cysteamine/PDITC/SPCE immunosensor;
detecting a snake specific venom by adding the species specific venom to the species specific GPH-GNP/cysteamine/PDITC/SPCE immunosensor and incubating in room temperature;
monitoring the reduction peak current variation after the venom binding using square wave voltammetry.

7. The method of claim 6, wherein the chemical linkers are Cysteamine HCL and 1,4-phenylene diisothiocyanate.

8. The method of claim 6, wherein the species in a snake species are Naja arabica, Walterinnesia aegyptia, Bitis arietans, Cerastes cerastes, Echis coloratus, Echis carinatus.

9. The method of claim 6, wherein, the modified electrode is Graphene-Gold Nanoparticle/cysteamine/PDITC/SPCE electrode.

10. A method of detecting a venom in a scorpion species simultaneously, comprising:
modifying a Graphene-Gold Nanoparticle (GPH-GNP)-Screen-Printed Carbon (SPCE) electrode with at least two chemical linkers in order to facilitate the immobilization of the antibodies through covalent binding;
washing the electrode with a N,N-dimethyl formamide and water mix and then ethanol to form a modified electrode;
conjugating an antibody of the snake species was added to the modified electrode to form a species specific GPH-GNP/cysteamine/PDITC/SPCE immunosensor;
detecting a snake specific venom by adding the species specific venom to the species specific GPH-GNP/cysteamine/PDITC/SPCE immunosensor and incubating in room temperature;
monitoring the reduction peak current variation after the venom binding using square wave voltammetry.

11. The method of claim 10, wherein the chemical linkers are Cysteamine HCL and 1,4-phenylene diisothiocyanate.

12. The method of claim 10, wherein, the species in a scorpion are Leiurus quinquestriatus and Androctonus crassicauda.

13. The method of claim 10, wherein, the modified electrode is Graphene-Gold Nanoparticle/cysteamine/PDITC/SPCE electrode.
